(19) United States
(12) Patent Application Publication
(10) Pub. No.: US 2004/0261790 A1
(43) Pub. Date: Dec. 30, 2004

(54) MOVING EMANATORS

(75) Inventors: Ashok V. Joshi, Salt Lake City, UT (US); Lawrence Domenick, Park City, UT (US)

Correspondence Address: FACTOR & LAKE, LTD, 1327 W. WASHINGTON BLVD., SUITE 5G/H, CHICAGO, IL, 60607, US

(73) Assignee: MICROLIN, L.C., Salt Lake City, UT (US)

(21) Appl. No.: 10/709,440

(22) Filed: May 5, 2004

Related U.S. Application Data
(60) Provisional application No. 60/483,833, filed on Jun. 30, 2003.

(51) Int. Cl. A61M 1/00; A61M 37/00; A61M 11/00; B05B 17/06; A61M 5/32; A61M 25/00

(52) U.S. Cl. 128/200.16; 128/200.14; 604/30

(57) ABSTRACT

A controlled sustained release fluid delivery device is disclosed which has enhanced fluid delivery. The device generally includes a housing having a fluid reservoir therein for holding a fluid, at least one opening in the housing for enabling fluid delivery out of the fluid reservoir and the housing, and structures for delivering the fluid, and retaining the fluid proximate an ambient environment. Once held there, the structure holding the fluid is vibrated so as to enhance the delivery of the fluid to the ambient environment. Other aspects are also disclosed.

### CROSS REFERENCE To RELATED APPLICATIONS

This application claims priority to U.S. Provisional Application No. 60/483,833, filed Jun. 30, 2003, which is incorporated herein by reference.

### BACKGROUND OF INVENTION

The present invention relates generally to the field of controlled (continuous or intermittent) release fluid delivery devices, and specifically to devices and methods for increasing the rate and dispersion of fluid from such devices.

Controlled-release fluid delivery devices have been used in the art to deliver a variety of fluids, including perfume, pesticides, insecticides, and other fluids for which prolonged aerosol delivery may be advantageous. These fluids provide a beneficial effect, generally upon release of the fluid, which can be extended as long as the time for delivery of the fluid can also be extended. To that end, numerous structures have been utilized to help deliver such fluids, including spray canisters, evaporation pools, wicking mechanisms, force-fed delivery devices and gravity-fed delivery devices, for example.

In order to enhance the delivery of fluid from these devices, some fluid delivery devices have added one or more structures for subjecting the fluid to vibrations, via piezoelectric surfaces. For example, a number of devices have used atomizers, ultrasonic vibrators, and vibrating plates to enhance the delivery of fluid. By adding piezoelectric vibration to the device in one way or another, these devices enable the enhanced evaporation and distribution of fluid into the surrounding environment.

One particularly useful structure for providing vibrations has been the use of piezoelectric materials. These piezoelectric materials comprise any number of known substances that have an electric polarity, either naturally or as a result of pressure on the substance. As such, when the piezoelectric substance is subjected to an electric field, it begins to vibrate. These piezoelectric structures can come in a variety of shapes and sizes.

All of the known fluid delivery devices, however, have required fluid to be present on the vibrating planar surfaces in a thin-film state in order to properly deliver that fluid.

Further, many of these devices have also required a complicated and often expensive delivery system to introduce a proper amount of fluid on the surface of the piezoelectric vibration elements for delivery.

It is therefore an object of this invention to demonstrate a simple and effective method for the enhanced delivery of a fluid using simple motion of a delivery mechanism (moving emanators) employing electric motors, electrochemical cells, magnetic devices, sonic waves or ultrasonic waves.

It is an additional object of this invention to enable this enhanced delivery with reliable and relatively inexpensive fluid delivery mechanisms.

It is also an additional object of this invention to show simple and effective design of one or multiple vibrating or moving wicks for enhanced fluid delivery by imparting motion using small a electric motor.

It is also an additional object of this invention to demonstrate the combination of delivering fluid out of reservoir on to a wick or emanator or capillary tubes and vibrating the wick or emanator or capillary tubes to dispense the fluid into the surrounding environment.

It is also an additional object of this invention to demonstrate the distinction between vibrating hard surfaces and vibrating or moving wicks or emanators or sponges or capillary tubes for dispension of fluid into the environment.

It is also an object of this invention to demonstrate the delivery of fluids into surrounding environment in a two-step process, namely first delivering the fluid out of fluid reservoir lining one or more of electrochemical or chemical gas generating force or gravity force or electrochemical or chemical water generating force or capillary force and then secondly, vibrating or moving the delivered fluid using either a piezoelectric device, magnetic device, electric mechanical vibrating device, sonic waves or a gas generating device. Such a vibrating device is associated with the delivery mechanism.

These and other objects will become apparent to one of ordinary skill in the art in light of the specification, drawings and claims appended hereto.

### BRIEF DESCRIPTION OF DRAWINGS

FIG. 1 is a schematic representation of a first embodiment of a fluid delivery device according to the principles of the present invention, incorporating a wick;

FIG. 2 is a schematic representation of a second embodiment of a fluid delivery device according to the principles of the present invention, wherein a wick is associated with a three-dimensional member;

FIG. 3 is a schematic representation of a third embodiment of a fluid delivery device according to the principles of the present invention, wherein a wick is associated with a hollow member;

FIG. 4 is a schematic representation of a fourth embodiment of a fluid delivery device according to the principles of the present invention, incorporating a porous pad;

FIG. 5 is a schematic representation of a fifth embodiment of a fluid delivery device according to the principles of the present invention, wherein a porous pad is associated with a three-dimensional member;

FIG. 6 is a schematic representation of a sixth embodiment of a fluid delivery device according to the principles of the present invention, wherein a porous pad is associated with a hollow member; and

FIG. 7 is a schematic representation of a seventh embodiment of a fluid delivery device according to the principles of the present invention, incorporating a motion element that imparts rotational motion to a fluid retaining member.

### DETAILED DESCRIPTION

While this invention is susceptible of embodiment in many different forms, there is shown in the drawings and will be described in detail, specific embodiments with the understanding that the present disclosure is to be considered as an exemplification of the principles of the invention and is not intended to limit the invention to the embodiments illustrated.

The present invention comprises an improved device for the delivery of fluid to an ambient environment. Generally, the principles described herein can be applied to a wide variety of fluid delivery devices. For clarity and brevity, however, several specific embodiments will be addressed below.

One preferred embodiment of controlled release fluid delivery device 10 is shown in FIG. 1 as comprising housing 12 having generally upward-facing opening 16 providing an aperture for access into fluid reservoir 14 within housing 12. Wick 18 is inserted into fluid reservoir 14 through opening 16, and placed in contact with a fluid that is contained therein. Wick 18 provides a means for transporting fluid out of fluid reservoir 14 via a capillary effect (as indicated by arrow F), and then retaining that fluid at or near the surrounding ambient air in order to allow the fluid to evaporate. To enhance the evaporation of the fluid from wick 18, wick 18 may additionally include, or be in communication with, means for imparting motion, such as vibration, in the form of a motion mechanism or motion element 20 (or a plurality of motion elements), which can increase both the rate of evaporation as well as providing a mechanical means for dispersal of the fluid. Together, these elements enable the efficient, delivery of a fluid from housing 12.

Generally, housing 12, fluid reservoir 14, opening 16 and wick 18 may comprise conventional structures known in the art. For example, housing 12 and fluid reservoir 14 can comprise a fluid delivery device such as the ones shown in U.S. Pat. Nos. 5,932,204, 6,045,055, and 6,042,704, the specifications of which are incorporated herein by reference. In such devices, housing 12 includes an outer wall within which fluid reservoir 14 is located for holding and retaining a fluid. The outer wall can be configured in numerous three-dimensional shapes, such as cylindrical, spherical, rectangular or square.

A number of different means for imparting motion, such as vibration, can be implemented via the motion element 20. As shown in FIG. 1, motion element 20 is in the form of a vibrator, which can comprise one or more vibrating elements (such as piezoelectric devices, vibrating electric motors, vibrating magnetic devices, magnetic motors, sonic waves, and gas generating cells) woven or otherwise associated with wick 18. Vibrating elements are associated with at least the portion of wick 18 exposed to the surrounding ambient air. Vibrating elements act as vibrating elements within wick 18, vibrating wick 18 a corresponding amount as they are placed under the influence of electric field. This vibration, in turn, helps to increase the rate at which fluid is dispersed from wick 18 to the surrounding environment.

Another preferred motion element 20 is shown in FIG. 2. Similar to FIG. 1, controlled-release fluid delivery device 10 still comprises housing 12 with fluid reservoir 14, generally upward-facing opening 16, and wick 18 protruding therefrom to facilitate delivery of fluid (as indicated by arrows F). In the embodiment shown in FIG. 2, however, device 10 also includes motion element 20 in the form of a three-dimensional member, such as a cylinder, around which wick 18 is associated. Although a cylindrical member is shown in the drawings, other three-dimensional structures could also be used, as would be known by one of ordinary skill in the art, with wick 18 being associated with at least one surface of the three-dimensional structure. The motion element 20—in this embodiment in the form of a cylinder—is preferably constructed from a vibrating material. In an alternate embodiment, however, the cylindrical or other geometrically shaped member may contain, or be in communication with, one or more vibrating elements to enable vibration of the cylindrical or other geometrically shaped member.

Wick 18 need only be associated with the motion element 20 in such a way as to enable the vibrations of motion element 20 to be transferred thereto. In the embodiment shown in FIG. 2, the wick 18 is preferably wound in a helical fashion around the cylindrically-shaped motion element 20 so as to maximize the contact surface between wick 18 and the motion element 20, as well as maximizing the area of wick 18 that is in turn exposed to the surrounding environment. Of course, alternative configurations could also be utilized as well.

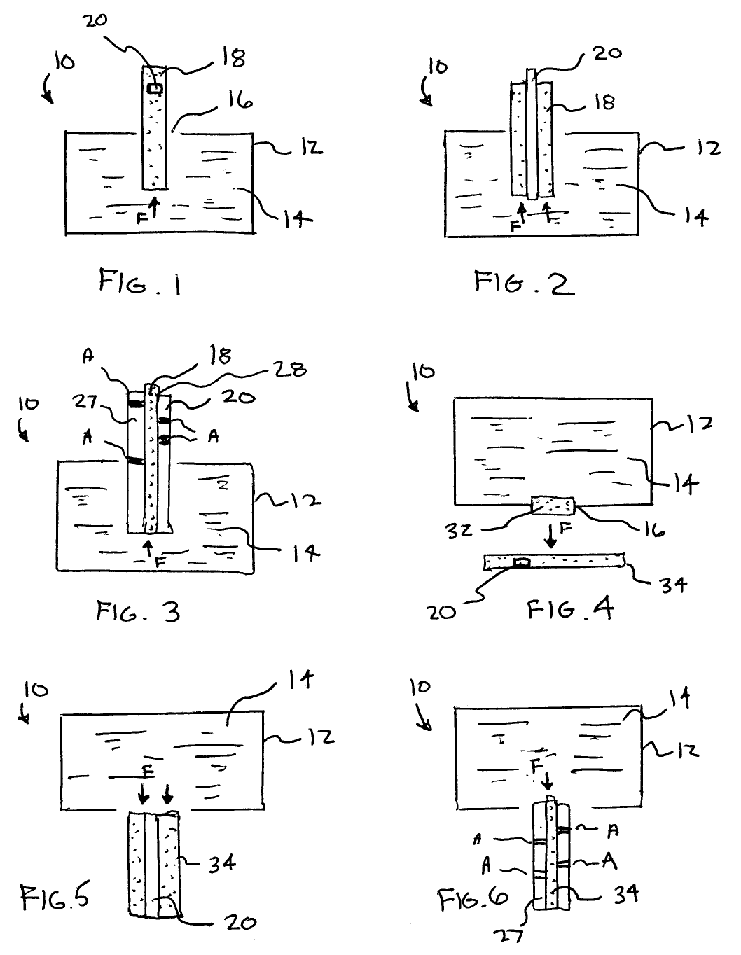

Still another preferred embodiment is shown in FIG. 3, in which motion element 20 comprises a hollow member, through which at least a portion of wick 18 is passed. In this embodiment, the motion element 20 comprises a three-dimensional shell 27 having at least one passage 28 therethrough. Shell 27 at least partially surrounds an open interior portion, or hollow space in the interior of shell 27. The shell 27 is preferably constructed from a vibrating component, or contains at least one vibrating component, enabling it to vibrate. Since a portion of wick 18 extends into shell 27 through passage 28, as the shell 27 vibrates, it helps to vibrate wick 18 also. Additionally, the shell 27 may include one or more apertures (A) to provide an avenue for the evaporation/dispersion of fluid from wick 18 as wick 18 is vibrated by the shell 27.

All of the above embodiments enable the delivery of a fluid via the capillary effect of wick 18 (as indicated by arrows F in FIGS. 1-3). Other methods of delivery could also be used to deliver fluid from fluid reservoir 14, of course, including forced-fluid delivery and gravity-fed fluid delivery. To illustrate other embodiments of the present invention, FIGS. 4-6 have been included to demonstrate the present invention as utilized in a gravity-fed or assisted fluid delivery device (the direction of fluid delivery indicated by arrows F in FIGS. 4-6).

As shown in FIG. 4, sustained-release fluid delivery device 10 is shown having housing 12 containing fluid reservoir 14, and generally downward-facing opening 16 for delivering fluid out of fluid reservoir 14. Additionally, fluid delivery device 10 includes porous plug 32 associated with opening 16 to help control the rate of fluid delivery out of fluid delivery device 10. Fluid is delivered out of fluid delivery device 10 through porous plug 32 and onto porous pad 34, instead of wick 18, which is positioned proximate opening 16. From porous pad 34, fluid may then be dispersed into the surrounding environment.

Porous pad 34 can comprise any of a number of structures that are porous and capable of receiving and retaining a fluid therein. For example, porous pad 34 could comprise a sponge, felt, absorbers, porous, plastics or ceramics. Regardless of the particular structure selected, porous pad 34 should be able to receive a fluid from fluid delivery device 10, and retain that fluid proximate the ambient environment.

In FIG. 4, porous pad 34 incorporates a motion element 20 in the form of at least one vibrator (such as a piezo device, electric motor, magnetic motor, gas generating cell, sonic wave device, or the like) associated therewith. Preferably, porous pad 34 includes a vibrator integrated into pad 34 itself. In such a configuration, when porous pad 34 is exposed to a fluid, it retains that fluid proximate the surrounding environment, where the fluid may subsequently be evaporated/dispersed. The vibrator, under the influence of electric field, vibrates and, in turn, vibrate porous pad 34 to increase the evaporation/dispersion of fluid from pad 34.

This delivery of fluid to the fluid retaining structure can be achieved through numerous ways using force such as using a conventional gas generating cell, electrochemical cell, wick or multiple wicks or multiple capillary tubes, gravity force or electro-mechanical force, or the like. Any single force or combination of these forces can be used to force the fluid out of cartridge or housing 12 on to a retainer porous sponge or wick.

FIG. 5 shows a similar embodiment to FIG. 4. In FIG. 5, however, porous pad 34 is associated with the external portion of a cylindrically-shaped motion element 20. As with the embodiment shown in FIG. 2, the motion element 20 comprises a three-dimensional structure either constructed of a vibrating material, or which contains at least one piezoelectric vibrator or electric motor or magnetic vibrator or gas generative device or sonic wave generator. Porous pad 34 is wrapped at least partially around the cylindrically-shaped motion element 20, so that, upon its vibration, porous pad 34 is also vibrated, increasing distribution of fluid therefrom.

Referring to FIG. 6, the motion element 20 comprises a three-dimensional shell 27 having at least one passage 28 therethrough. Shell 27 at least partially surrounds an open interior portion, or hollow space in the interior of shell 27. The shell 27 is preferably constructed from a vibrating component, or contains at least one vibrating component, enabling it to vibrate. Since a portion of wick 18 extends into shell 27 through passage 28, as the shell 27 vibrates, it helps to vibrate wick 18 also. Additionally, the shell 27 may include one or more apertures (not shown) to provide an avenue for the evaporation/dispersion of fluid from wick 18 as wick 18 is vibrated by the shell 27. In this embodiment, delivery of the fluid is assisted by gravity force. The shell 27, as in the embodiment shown in FIG. 3, is either constructed at least partially from a piezoelectric material, or contains one or more vibrating elements. As such, when the shell 27 is vibrated, retaining pad 34 is also vibrated, aiding the release of fluid out of apertures (A).

Regardless of the particular embodiment selected for a particular application, the operation of the device is substantially similar. In operation, enhanced-release fluid delivery device 10 begins delivery of fluid out of housing 12.

In the device configuration having wick 18, fluid from within fluid reservoir 14 is drawn out of housing 12 using the capillary effect of wick 18. In the device configuration having retaining pad 34, fluid is delivered out of housing 12 (force fed or gravity fed, for example) and onto retaining pad 34. In either configuration, and after delivery of the fluid, the fluid is retained proximate the ambient atmosphere.

Once delivery of the fluid is complete, motion element 20 is activated to, in turn, impart motion to the structure in which the fluid is retained. As the structure is moved, the movement increases both the rate of evaporation and the dispersion of fluid from the device.

With any of the embodiments described herein, the means for retaining the fluid, such as a retaining member in the form of a wick or capillary tube, may include a plurality of retaining members. Further, in the case of a wick or a capillary tube, these elements are also included in the means for delivering the fluid out of the fluid container or reservoir.

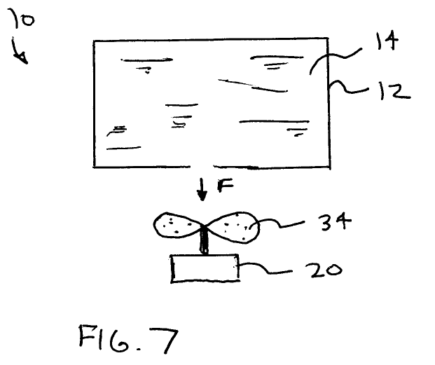

Referring now to FIG. 7, an embodiment of the present invention is shown wherein the motion element 20 is in the form of an electric motor, wherein it can be used to impart rotational motion to a fluid retainer, such as the porous pad 34. The rotational motion of the pad 34 tends to increase the evaporation/dispersion of fluid from the pad 34. In this type of configuration, the pad 34 can be further configured in a blade-like shape, such as that of a fan blade, and rotated by the electric motor. The pad 34 can alternatively be disposed as a coating or layer over a fan blade of an electric motor to provide a more rigid structure. In lieu of porous pad 34, other fluid retainer members or materials could be implemented in this embodiment, including other materials exhibiting fluid retaining properties and other materials that can be applied to a fan blade surface as a coating or integrated therein. Fluid may be delivered to the rotating pad 34 via a number of mechanisms, including gravity feeding or force feeding mechanisms, for example. In an embodiment, the rotating porous pad 34 can be in fluid communication with the fluid reservoir such that fluid is transported to the porous pad 34 via a wicking process, capillary effect, absorption, or the like.

It should be noted that numerous configurations for imparting motion to the porous pad 34 or other fluid retaining structure are contemplated that would be apparent to one of ordinary skill in the art, and such structures are considered suitable for implementation in accordance with the principles of the present invention.

As should be readily apparent from the foregoing description, and in accordance with the principles of the present invention, a controlled-release fluid delivery device having enhanced fluid delivery is provided wherein the device incorporates a fluid reservoir for holding a fluid, means for delivering the fluid out of the fluid reservoir, means for retaining the fluid delivered out of the fluid reservoir proximate an ambient environment, and means for imparting motion to the retaining means to enhance emanation of the fluid to the ambient environment.

While specific embodiments have been illustrated and described, numerous modifications may come to mind without significantly departing from the spirit of the invention, and the scope of protection is only limited by the scope of the accompanying Claims.

1. A controlled-release fluid delivery device having enhanced fluid delivery, comprising:
a housing having a fluid reservoir therein for holding a fluid;
at least one opening in the housing for enabling fluid delivery out of the fluid reservoir and the housing;
means for delivering the fluid out of the fluid reservoir;
means for retaining the fluid, delivered out of the fluid reservoir, proximate an ambient environment; and
means for imparting motion to the retaining means to, in turn, enhance the delivery of the fluid to the ambient environment.

2. The delivery device according to claim 1, wherein the means for delivering a fluid and the means for retaining the fluid are integrated.

3. The delivery device according to claim 2, wherein the means for delivering the fluid and the means for retaining the fluid is selected from the group consisting of a capillary tube, a plurality of capillary tubes, a wick, and a plurality of wicks.

4. The delivery device according to claim 3, wherein the means for delivering the fluid and the means for retaining the fluid are in communication with the fluid within the fluid reservoir and are in communication with the ambient environment.

5. The delivery device of claim 1, wherein the means for delivering the fluid is selected from the group consisting of a gas generating cell, a water generating cell, an electrochemical cell, gravity force, a wick, a plurality of wicks, a capillary tube, a plurality of capillary tubes, and combinations thereof.

6. The delivery device according to claim 1, wherein the means for imparting motion is selected from the group consisting of a piezoelectric device, an electric motor, a vibrating motor, a sonic wave producing device, a gas producing cell, and combinations thereof.

7. The delivery device according to claim 6, wherein the means for imparting motion is integrated with the means for retaining the fluid.

8. The delivery device according to claim 6, wherein the means for imparting motion comprises a vibrating member configured in a three-dimensional shape having at least a portion capable of imparting vigorous motion to the means for retaining fluid.

9. The delivery device according to claim 6, wherein the means for imparting motion comprises a vibrating member comprising a cylinder having a circumference adjacent to which the means for retaining the fluid is disposed.

10. The delivery device according to claim 1, wherein the means for imparting motion comprises a hollow member defining an interior portion, the hollow member having at least a portion that is capable of vibrating, wherein the means for retaining the fluid is at least partially disposed within the interior portion of the hollow member.

11. The delivery device according to claim 10, wherein the hollow member comprises a tube having at least one aperture therein, and the means for retaining the fluid is threaded through the tube so as to be surrounded thereby.

12. The delivery device according to claim 1, wherein the means for delivering the fluid comprises at least one opening within the fluid reservoir positioned in a generally downward-facing direction, and the means for retaining the fluid comprises at least one porous member positioned proximate the at least one opening.

13. The delivery device according to claim 12, wherein the at least one opening further includes means for controlling a rate of fluid flow therethrough.

14. The delivery device according to claim 12, wherein the means for imparting motion comprises at least one motion element in communication with the porous member and selected from the group consisting of a piezoelectric device, an electric motor, a magnetic motor, a sonic wave generator, a gas generating cell, and combinations thereof.

15. The delivery device according to claim 14, wherein the motion element is integrated into the porous member.

16. The delivery device according to claim 14, wherein the motion element is disposed adjacent to the porous member.

17. The delivery device according to claim 14, wherein the motion element comprises at least one surface adjacent the porous member that is capable of vibrating.

18. The delivery device according to claim 1, wherein the means for delivering the fluid comprises at least one opening within the fluid reservoir and a device that forces the fluid out of the fluid reservoir through the at least one opening, and the means for retaining the fluid comprises a porous member positioned proximate the at least one opening.

19. The delivery device according to claim 18, wherein the means for imparting motion to the means for retaining the fluid is selected from the group consisting of a piezoelectric device, an electric motor, a magnetic motor, a sonic wave generator, a gas generating cell, and combinations thereof.

20. The delivery device according to claim 19, wherein the means for delivering a fluid and the means for retaining the fluid are integrated.

21. The delivery device according to claim 18, wherein the means for imparting motion to the means for retaining the fluid comprises a motion element disposed adjacent the means for retaining the fluid.

22. The delivery device according to claim 18, wherein the means for imparting motion to the means for retaining the fluid comprises at least one surface disposed adjacent the means for retaining the fluid, wherein at least a portion of the at least one surface being capable of movement.

23. The delivery device according to claim 18, wherein the means for imparting motion comprises a hollow member having an interior portion, at least a portion that is capable of vibrating, and at least one aperture therein, wherein the means for retaining the fluid is at least partially positioned within the interior portion of the hollow member.

24. A controlled release fluid delivery device comprising:
a housing having a fluid reservoir for holding a fluid;
means for delivering the fluid out of the fluid reservoir; and
means for vibrating the means for delivering the fluid.

25. The delivery device of claim 24, wherein the means for delivering the fluid is selected from the group consisting of a wick, a capillary tube, a sponge, a porous plug, a tube, and combinations thereof.

26. The delivery device of claim 24, wherein the means for imparting motion is selected from the group consisting of a piezoelectric device, an electric motor, a magnetic motor, a sonic wave generator, a gas generating cell, and combinations thereof.

27. A method of increasing the emanation of a fluid to an ambient environment from a controlled-release fluid delivery device, the method comprising the steps of:
delivering a fluid from a fluid reservoir contained within a housing out of at least one opening in the housing;
retaining the delivered fluid in a retaining member proximate an ambient environment; and
imparting motion to the retaining member to increase emanation of the fluid to the ambient environment.

28. A method of increasing the delivery of a fluid from a controlled-release fluid delivery device, the method comprising the steps of:
delivering a fluid from a fluid reservoir contained within a housing out of at least one opening in the housing;
retaining the delivered fluid in a porous pad proximate an ambient environment; and
imparting motion to the porous pad to increase the emanation of the fluid to the ambient environment.

29. A controlled-release fluid delivery device having enhanced fluid delivery, comprising:
a housing having a fluid reservoir therein for holding a fluid;
at least one opening in the housing for enabling delivery of the fluid out of the fluid reservoir and the housing;
a fluid delivery mechanism in communication with the fluid to facilitate delivery of the fluid out of the fluid reservoir;
a fluid retainer disposed proximate an ambient environment and in communication with the fluid delivered out of the fluid reservoir to retain the fluid; and
a motion mechanism in communication with the fluid retainer to impart motion thereto and thereby enhance the delivery of the fluid to the ambient environment.

30. A controlled-release fluid delivery device comprising:
a housing having a fluid reservoir therein for holding a fluid and at least one opening in the housing for enabling delivery of the fluid out of the fluid reservoir;
a fluid retainer disposed proximate an ambient environment and in communication with the at least one opening, the fluid retainer retaining the fluid delivered out of the fluid reservoir; and
a motion element in communication with the fluid retainer to impart motion thereto and thereby enhance the delivery of the fluid to the ambient environment.

31. The fluid delivery device of claim 30, wherein the motion element is a motor that imparts rotational motion to the fluid retainer.

32. The fluid delivery device of claim 31, wherein the fluid retainer has a blade-like shape.

33. The fluid delivery device of claim 31, wherein the fluid retainer is a coating on a blade in communication with the motor.
